(19) United States
(12) Patent Application Publication
(10) Pub. No.: US 2014/0316119 A1
(43) Pub. Date: Oct. 23, 2014

(54) COMPOSITION FOR IMPROVING BIOMETABOLISM PARAMETER

(71) Applicants: Koji Yamada, Itami-shi (JP); Noriko Hayashi, Itami-shi (JP); Takako Yamada, Itami-shi (JP)

(72) Inventors: Koji Yamada, Itami-shi (JP); Noriko Hayashi, Itami-shi (JP); Takako Yamada, Itami-shi (JP)

(73) Assignees: MATSUTANI CHEMICAL INDUSTRY CO., LTD., Itami-shi, Hyogo (JP); RARE SUGER FOODS, LLC., Marugame-shi, Kagawa (JP)

(21) Appl. No.: 14/342,122

(22) PCT Filed: Aug. 8, 2012
(86) PCT No.: PCT/JP2012/070226

§ 371 Date: May 1, 2014

(30) Foreign Application Priority Data
Sep. 6, 2011 (JP) 2011-194037

(51) Int. Cl. C07H 3/02

(52) U.S. Cl. CPC C07H 3/02
USPC 536/1.11

(57) ABSTRACT

A composition for improving biometabolism parameters is provided. The composition contains D-psicose as an active ingredient, and is continuously ingested in a dose of 5 g or more per intake in terms of D-psicose to lower biometabolism parameters represented by a uric acid level and/or a liver function indicator component level. γ-GTP showed a significant decrease after 4 weeks from 15-g ingestion. γ-GTP and ALP showed a significant decrease after 4 weeks and 2 weeks, respectively, from the ingestion of D-psicose continuously administered three times each day in a dose of 5 g or more per intake.

### TECHNICAL FIELD

The present invention relates to a composition that contains D-psicose as an active ingredient for improvement of biometabolism parameters.

As used herein, the terms “liver function indicator component level” and “uric acid level” will be collectively referred to as “biometabolism parameters”.

### BACKGROUND ART

The human liver is the largest glandular organ in the body, essential for survival. The liver is made up of a total of about three-hundred billion cells. The liver is involved in a wide range of biological responses with its characteristic functions, including metabolism and storage of nutrients, detoxification, phagocytosis of foreign objects and microorganisms, and regulation of blood circulation.

Various indicators are available for such liver functions, such as γ-GTP (γ-glutamyl transpeptidase), ALT (alanine aminotransferase), AST (aspartate aminotransferase), and ALP (alkaline phosphatase in the serum).

γ-GTP is an enzyme associated with drug metabolism in liver microsomes. The γ-GTP level increases in response to cholestasis or alcohol- or drug-induced liver damage, and can thus be used as a test indicator for the screening of liver and biliary functions.

ALT, a transaminase that uses pyridoxal phosphate as coenzyme, also represents a test indicator of liver function as does γ-GTP. ALT is particularly abundant in liver, and is less distributed in other organs compared to AST. With its rise being specific to liver damage, ALT is used as an indicator of hepatitis progression.

AST is a transaminase that uses pyridoxal phosphate as coenzyme, and represents a test indicator of liver function as with the case of ALT. AST is released into the blood when the liver cells are damaged. AST levels also increase when tissues and cells such as skeletal muscle, cardiac muscle, and red blood cells are destroyed.

Increased ALP concentrations in the serum can be interpreted as increased synthesis associated with regeneration after organ necrosis or destruction. The ALP level can thus be also used as an indicator in liver function evaluation.

In actual practice, the determination of liver damage takes a comprehensive approach that takes into account the fluctuation of multiple parameters, rather than that of a single parameter. Among such parameters, γ-GTP and ALT levels, in particular, are often given high priority in the determination of liver function.

A uric acid level also represents an important biometabolism parameter. A blood uric acid concentration above 7 mg/dL indicates hyperuricemia.

D-psicose has low calories (Patent Document 1, and Non-Patent Documents 1 and 2), and is hardly converted into energy. In addition, 70% of the D-psicose ingested is directly absorbed but excreted into urine. The remaining 30% reportedly reaches the large intestine, and excreted into feces essentially without being utilized by enteric bacteria. Functionally, D-psicose has been reported to have postprandial glucose level suppressing effect (Non-Patent Document 2). The mechanisms by which D-psicose exhibits such effect are mainly through inhibition of sugar breakdown, and promotion of glucose uptake in the liver. Other reported characteristics of D-psicose include fat reduction (Patent Document 2). However, the mechanism of action remains elusive.

Alkali isomerization is the industrial sugar isomerization method described in Patent Document 3.

### CITATION LIST

### Patent Documents

Patent Document 1: JP-A-2007-51137
Patent Document 2: JP-A-2010-18528
Patent Document 3: WO2010/113785
Patent Document 4: JP-A-6-125776

### Non-Patent Documents

Non-Patent Document 1: Metabolism. 59(2), 206-214, 2010
Non-Patent Document 2: J Nutr Sci Vitaminol. 48:77-80.
Non-Patent Document 3: J. Am. Chem. Soc. 1955. 77. 3323-3325
Non-Patent Document 4: Biochemistry. 16 (10):2169-75, 1977

### SUMMARY OF THE INVENTION

### Problems that the Invention is to Solve

Although the functions of D-psicose have been elucidated to some extent, the liver fat reduction described in Patent Document 2 is the only report concerning its liver-associated function. Further, there is no report that describes the effect of D-psicose on the uric acid level. It also has not been elucidated whether ingestion of D-psicose has any effect on various liver indicators and if so, in what amount.

It is accordingly an object of the present invention to present an effective dose or effective period with which the uric acid level and the levels of the liver function indicators γ-GTP, AST, ALT, and/or ALP can be reduced.

There is also a strong need for the development of a drug that can effectively lower the uric acid level or treat liver disease, and a health beverage and food, or an animal feed that can lower the uric acid level, and prevent or treat liver function damage through daily ingestion. An object of the present invention, then, is to provide an agent for lowering the uric acid level or for protecting or improving liver function, a beverage and food, a feed, and a medicinal product for lowering the uric acid level or for protecting or improving liver function, and a beverage and food additive having uric acid level lowering effect or liver function protecting or improving effect.

### Means for Solving the Problems

The present inventors conducted intensive studies to achieve the foregoing objects, and found that D-psicose has the effect to lower the biometabolism parameters (γ-GTP, AST, ALT, ALP, and uric acid level) of uric acid and liver function indicator components. The present invention as a lowering agent was completed after determining the effective dose and the effective period of such D-psicose.

Specifically, the present invention provides biometabolism parameter improving agents (γ-GTP, AST, ALT, ALP, and uric acid level lowering agents) that contain D-psicose as an active ingredient.

The gist of the present invention includes the following compositions (1) to (6) for improving biometabolism parameters.

(1) A composition comprising D-psicose as an active ingredient,

the composition being continuously ingested in a dose of 5 g or more per intake in terms of D-psicose to lower biometabolism parameters represented by a uric acid level and/or a liver function indicator component level.

(2) The composition according to (1), wherein the D-psicose contained as an active ingredient is an alkali-isomerized product of glucose, fructose, and/or isomerized sugar.

(3) The composition according to (1) or (2), wherein the liver function indicator component is γ-GTP, AST, ALT, and/or ALP.

(4) The composition according to any one of (1) to (3), wherein the composition is ingested in a daily dose of 15 g/day for 2 weeks in terms of D-psicose.

(5) The composition according to any one of (1) to (4), wherein the composition is a liver function protecting agent or a liver function improving agent.

(6) The composition according to any one of (1) to (4), wherein the composition is a beverage, a food, a feed, a medicinal product, a quasi drug, an oral composition, or a cosmetic for protecting or improving liver function, or a beverage or food additive having a liver function protecting or improving effect.

### Advantage of the Invention

The present invention determined the effective dose and the effective period for lowering the uric acid level and the liver function indicators γ-GTP, AST, ALT and/or ALP. The effective dose and the effective period were found to be continuous administration of 5 g or more per intake, preferably a daily dose of 15 g/day for 2 weeks, as demonstrated in Examples.

The present invention also can provide a drug that can effectively lower the uric acid level or effectively treat liver disease, and a health beverage and food or an animal feed that can lower the uric acid level or prevent or treat liver function damage through daily ingestion. Specifically, the present invention can provide an agent for lowering the uric acid level or for protecting or improving liver function, a beverage and food, a feed, and a medicinal product for lowering the uric acid level or for protecting or improving liver function, and a beverage and food additive having uric acid level lowering effect or liver function protecting or improving effect.

### BRIEF DESCRIPTION OF THE DRAWINGS

FIG. 1-1 is a diagram representing changes in γ-GTP and ALP of Example 1.

FIG. 1-2 is a diagram representing changes in the uric acid level of Example 1.

FIG. 2 is a diagram representing changes in γ-GTP, ALT, and ALP of Example 2.

### MODE FOR CARRYING OUT THE INVENTION

In the present invention, γ-GTP, AST, ALT, and/or ALP are used as liver function indicator components, and the levels of these liver function indicator components, and a uric acid level are collectively referred to as biometabolism parameters.

The specific functions of the D-psicose-containing material of the present invention are the uric acid level lowering effect, and the effect of lowering the liver function indicators γ-GTP, AST, ALT, and ALP. These effects offer protection of liver cells and the like.

The D-psicose of the present invention may be one that is enzymatically or chemically produced from D-fructose, or indirectly from D-glucose through epimerization, or may be one that is extracted from plants, and may be purified completely, or may contain trace amounts of impurities present after the production. For example, the D-psicose of the present invention can be prepared relatively easily by using the technique that uses epimerase (see, for example, Patent Document 4). The D-psicose of the present invention also may be chemically produced (Non-Patent Document 3).

The D-psicose solution obtained may be purified using a method such as deproteination, decolorization, and desalting, as required, and may be concentrated to be obtained as a syrup-like D-psicose product. The product may be fractionated and purified by column chromatography, upon which a standard preparation with a purity of 99% or more can easily be obtained. The product D-psicose can directly be used as a monosaccharide.

The D-psicose also may be used as a mixed sugar, for example, a mixed sugar containing D-glucose, D-fructose, or other rare sugars (e.g., allose).

The D-psicose may be used as a D-psicose and/or a derivative thereof.

The D-psicose derivatives used in the present invention are described below. A derivative is a compound obtained after the conversion of a starting compound through a chemical reaction that changes the molecular structure. There is a wide variety of D-psicose derivatives along with derivatives of other hexoses. Non-limiting examples include amino sugars (sugars substituted with a NH₂ group at an OH group of the sugar molecule; glucosamine, chondrosamine, and glycoside). In the case of monosaccharide physiological effects, the main mechanism of action depends on the specificity to enzyme, and the sugar conformation is particularly important. For example, fructose is the main substrate of the phosphorylation by the fructokinase enzyme in liver. This enzyme also phosphorylates D-tagatose and D-psicose, C-3 isomers of fructose, but their phosphorylation rates depend on the conformation (Non-Patent Document 4). The reaction rate of fructokinase is affected particularly by the trans/axial relationship of the 2-, 3-, 4-, and 5-position carbon atoms. It can thus be speculated that the effect of the present invention can be sufficiently obtained even with the derivatives, provided that the conformation is maintained to some extent.

The effective dose of D-psicose is 2.5 g to 20 g per intake. The effective dose may be decided depending on how much the biometabolism parameters need to be lowered, whether the body weight is within the standard body weight range, and the like. Preferably, the effective dose is typically 5 g or more per intake. In Example 1, the dosage is 5 g per intake, three times each day. In Example 2, D-psicose was ingested in a dose of 15 g at once, instead of the three divided doses of Example 1. The ingestion period can be reduced in this case.

The ingestion period of D-psicose is about 1 week, preferably 2 weeks, more preferably longer than 2 weeks.

The D-psicose used in the present invention may have any form, including a solid form such as a powder, a fine powder, a granule, a crystal, and a tablet, and a form of an aqueous solution or a solution. The method of production is not particularly limited either. It is also possible to contain non-sweet or non-taste components such as a bulking agent and a carrier, provided that such addition does not interfere with the purposes of the present invention.

The present invention also provides a beverage and food obtained using the D-psicose-containing material of the present invention, particularly materials with functions.

The beverage and food of the present invention encompasses a range of foods that require sweetness in general, including drinks, candies, frozen desserts, yogurts, and chocolates. Other examples of products with the imparted sweetness include medicinal products and oral compositions.

For example, the present invention can be advantageously used as a sweetener for various flavor enhancers such as soy sauce, a soy sauce powder, miso, a miso powder, moromi, hishio, furikake, mayonnaise, dressing, vinegar, sanbaizu (vinegar mixture), a sushi vinegar powder, a taste enhancer for Chinese food, tentsuyu (dipping sauce for tempura), mentsuyu (dipping sauce for noodles), sauce, ketchup, sauce for grilled meat, curry block, a stew powder, a soup powder, a soup stock powder, a combination flavor enhancer, mirin (sweet sake seasoning made from rice), shin-mirin (sweet sake seasoning made from coarse cereals), table sugar, and coffee sugar. Other possible advantageous uses include taste improvers, and quality improvers.

The present invention also can be used as an additive for various foods and beverages, including Japanese confectioneries such as senbei, arare, okoshi, rice cake, manju, uiro, red bean pastes, yokan, mizuyokan, kingyoku, jelly, castella, and candies; Western confectioneries such as bread, biscuits, crackers, cookies, pies, pudding, butter cream, custard cream, profiterole, waffles, sponge cake, doughnuts, chocolates, chewing gums, caramels, and candies; frozen desserts such as icecream and sherbet; syrups such as syruped fruits and korimitsu (syrup for shaved ice); pastes such as flower pastes, peanut pastes, and fruit pastes ; processed fruit and vegetable products such as jam and marmalade, syrup preserves, and sugar preserves; processed cereal products such as bread products, noodles, cooked rice products, and artificial meat; pickled vegetables such as fukujinzuke, bettarazuke, senmaizuke, and rakkyozuke; pickles powder products such as a takuanzuke powder and a hakusaizuke powder; animal products such as ham and sausage; fish products such as fish ham, fish sausage, kamaboko, chikuwa, and tempura; delicacies such as sea urchin, squid shiokara, sukonbu, sakisurume, and dried pufferfish with mirin; tsukudani products made from dried layer seaweed, sansai, dried shredded squid, small fish, and shellfish; side dishes such as nimame, potato salad, and a konbu roll; dairy product; bottled and canned products of fish meat, meat, fruits, and vegetables; alcohol products such as gouseishu (sake with additives), fruit wine, Western liquors, and liqueur; soft drinks such as coffee, hot chocolate, juice, carbonated drinks, lactic drinks, and lactobacillus beverages; premix powders such as a pudding mix, and a hotcake mix; and instant beverages and foods such as instant juice, instant coffee, instant red bean soup, and instant soup.

Specific examples of functional drinks include carbonated drinks such as coke, sports drinks, fruit juice, milk drinks, and tea drinks. Carbonated drinks, which can cause obesity when consumed in large quantities, represent a particularly desirable target.

The foods and beverages may be used as functional foods, dietary-supplements, or foods with health claims. The form of food is not particularly limited. For example, such food may be produced by using ordinary method, using materials such as proteins (e.g., milk proteins, soybean proteins, and egg albumin of high nutritional quality with a good balance of amino acids), degraded products of these proteins, egg white oligopeptides, soybean hydrolysate, and mixtures of amino acids alone. The foods and beverages may also be used, for example, in the form of a soft capsule, and a tablet.

Examples of the dietary-supplements or functional foods include processed products containing materials such as sugar, fat, trace element, vitamins, chemical agents, and flavors. Examples of such products include liquid food, defined formula diet, elemental diet, energy drink, capsule formulation, and enteral nutrient. These products, including food and beverages such as sports drinks, and nutritional supplement drinks may further contain nutritional additives and compositions, such as amino acids, vitamins, and minerals, and additives such as spice, flavor, and dye to enhance the nutritional balance and flavor.

The composition of the present invention may be used for all products that require the liver function indicator and/or body fat lowering effect. Examples of such products include common foods, foods for health use, clinical nutrition products, food materials, food materials for health use, clinical nutrition food materials, food additives, food additives for health use, clinical nutrition food additives, drinks, drinks for health use, clinical nutrition drinks, drinking water, drinking water for health use, clinical drinking water, drugs, pharmaceutical raw materials, feeds, and feeds for livestock and/or wild animals under treatment.

For food applications, the composition of the present invention may be used directly in a composition form, or in the form of a preparation, which may be of a form diluted in oil or the like, an emulsion form, or a form containing a carrier commonly used in food industry. The drinks may be non-alcohol drinks or alcohol drinks. Examples of the non-alcohol drinks include carbonated drinks, noncarbonated drinks such as fruit juice and nectar drink, soft drinks, sports drinks, tea, coffee, and hot chocolate. Examples of the alcohol drinks include common alcohol products, such as medicated liquor, chuhai, umeshu, beer, low-malt beer, and malt-free, beer-like alcoholic beverages.

For use as a food material or a food additive intended to improve the biological functions, the composition of the present invention may be used in the form of a solid agent such as a tablet, a capsule formulation, or a powder and a granule dissolved in a drink or the like, or in the form of a semi-solid such as a jelly, a liquid such as drinking water, or a high-concentration solution that is diluted before use.

The composition of the present invention may be appropriately added to food to provide a healthy diet or an invalid diet intended to improve biological functions. These may be appropriately mixed with additional components, such as vitamins, carbohydrates, dyes, and flavors commonly added to food. The food products may be ingested in any form, including a liquid form, and a solid form. Further, the composition of the present invention may be ingested in the form of a soft capsule formulation prepared by encapsulating the composition with gelatin or the like. Such capsules may be made of a gelatin coating prepared, for example, by dissolving the raw material gelatin by addition of water, and adding a plasticizer (such as glycerine, and D-sorbitol) to the gelatin solution.

The composition of the present invention is applicable to feeds for domestic animals, poultry, and pets. For example, the composition of the present invention may be mixed with dry dog foods, dry cat foods, wet dog foods, wet cat foods, semi-moist dog foods, poultry feeds, and feeds for domestic animals such as cows and pigs. Such feed itself may be prepared according to an ordinary method.

The therapeutic agent and the preventive agent may be used for non-human animals, including domestic mammals such as cows, horses, pigs, and sheep, poultry such as chicken, Japanese quail, and ostrich, pets such as reptiles, birds, and small mammals, and cultured fish.

The drug that takes advantage of the effects of the composition of the present invention may be used either alone, or may be mixed with a suitable additive such as a common excipient, a stabilizer, a preservative, a binder, and a disintegrant, and prepared into a suitable dosage form such as a liquid, a granule, a subtle granule, a powder, a tablet, a capsule formulation, a ball, an ointment, an adhesive skin patch, an epipastic, a spray, and an injection. These may be administered orally, transnasally, percutaneously, or intravenously.

A medicinal organic or inorganic solid, semi-solid, or liquid carrier, a solubilizer, or a diluent suited for oral administration, transnasal administration, transdermal administration, or intravenous administration may be used to prepare the composition of the present invention as a drug. Examples of the carriers usable for the drug containing the composition of the present invention include water, gelatin, lactose, starch, magnesium stearate, talc, animal and vegetable oils, benzyl alcohol, gum, polyalkylene glycol, petroleum resin, coconut oil, lanolin, and all other carriers used for medicinal applications. It is also possible to appropriately use a stabilizer, a wetting agent, and an emulsifier, and other adjuvant agents such as salts added to change osmotic pressure, or maintain the appropriate pH of a compounding agent.

Use of a soluble film has become common in the preparation of products such as cosmetics. For example, an edible soluble film has been used as a flavored film for purposes such as refreshing, and bad breath prevention. Examples of other possible applications that have been proposed include a moisturized cosmetic film produced as a mask, or as an emulsion by being dissolved in water. Potential use as a plaster containing an anti-inflammatory agent or the like also has been investigated. JP-A-2007-91696 proposes a soluble film that has excellent solubility and film characteristics, preferred for use as a wrapping material for food and products such as medicinal products, or as a food or medicinal product carrier for keeping the active ingredients. The composition of the present invention thus has use as a medicinal product, a quasi drug, or a cosmetic.

The present invention is described below in greater detail using Examples. It should be noted that the present invention is in no way limited by the following Examples.

Clinical trials were performed with ethics committee approval to examine the effects of D-psicose as a composition for improving biometabolism parameters.

### EXAMPLE 1

### <Experiment Methods>

Increasing incidence of lifestyle-related diseases, diabetes in particular, has been a problem in Japan. Clinical trials were conducted with recruited diabetic subjects, and biometabolism parameter changes due to D-psicose were investigated. The recruited subjects consisted of diabetes borderline subjects (12 subjects with fasting glucose levels of 110 to 126 mg/dl), and diabetes patients (6 type 2 diabetes patients undergoing drug treatment) (all subjects, males and females, were at least 20 years of age at the time of informed consent). Five grams of D-psicose was ingested per meal for 12 weeks. The D-psicose was purchased from Izumoring Co., Ltd. The subjects were tested after 0, 2, 4, 8, and 12 weeks from the start of the ingestion, and after 4 weeks from the end of the ingestion period (week 16, post-ingestion observation). The subjects were fasted after the dinner served on the day before the testing date. The subjects were measured for γ-GTP, ALT, ALP, and a uric acid level using the service of BML Inc. A paired t-test was used for statistical comparison before and after the ingestion. In the figure, significant difference is given by *:0.05<P or **:0.01<P.

### <Results>

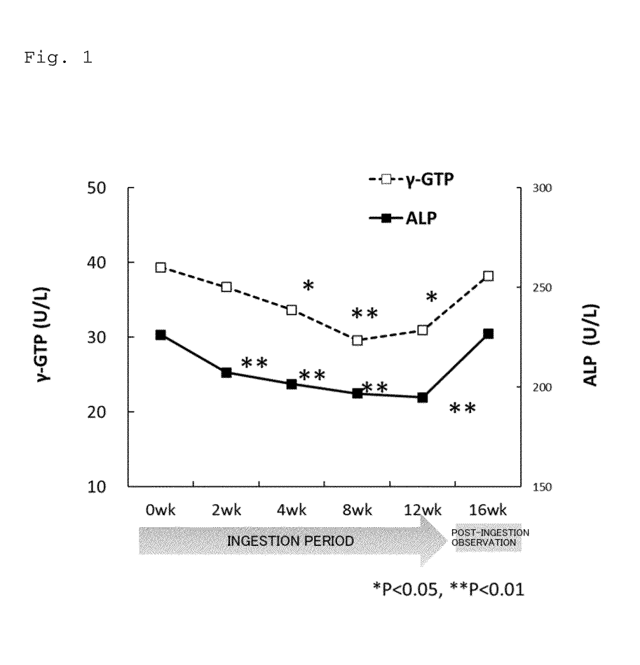

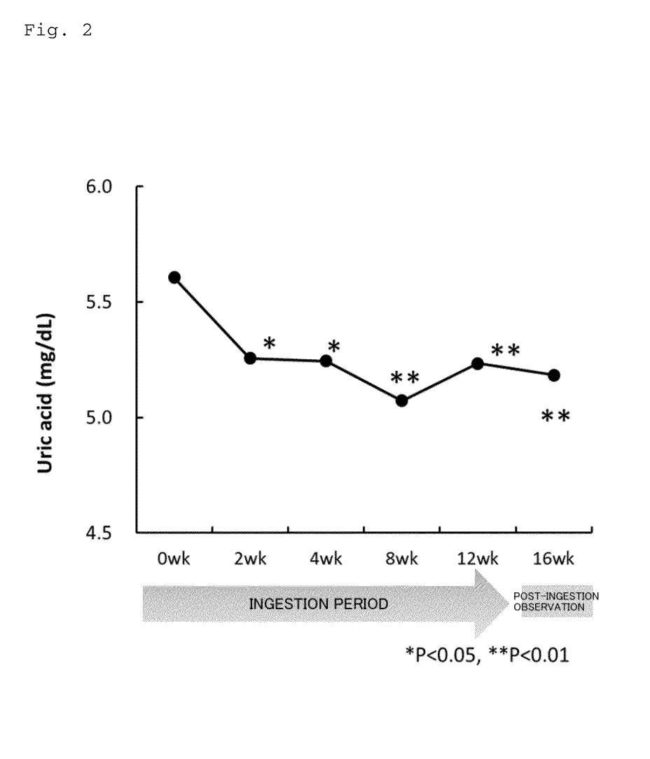

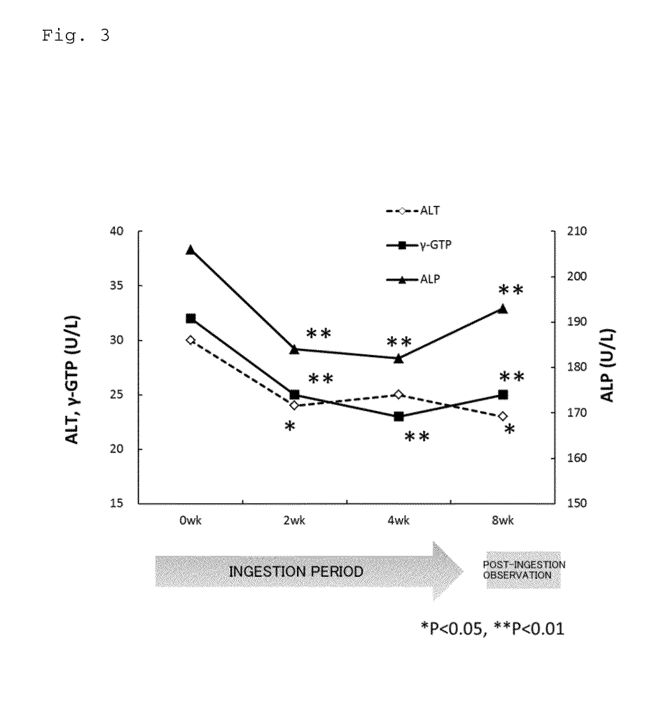

The results are shown in FIG. 1-1 (γ-GTP and ALP), and FIG. 1-2 (uric acid level). γ-GTP significantly decreased after 4 weeks from the ingestion, whereas ALP showed a significant decrease after 2 weeks from the ingestion.

These results demonstrated that the repeated ingestion of D-psicose (about 5 g/intake) over the time period of about 2 to 4 weeks can clearly decrease γGTP and ALP, and improve the biological function parameters (liver functions in particular) in humans. Stratified analyses of the diabetes borderline patients and the diabetes patients revealed a significant decrease (P<0.01) in γ-GTP after 4 weeks from the ingestion in the diabetes borderline patients, and after 8 weeks in the diabetes patients. ALP significantly decreased (P<0.05) after 2 weeks from the ingestion in both the diabetes borderline patients and the diabetes patients. These results demonstrated that D-psicose was effective for the diabetes borderline patients and the diabetes patients. It can be inferred from the extent of the decrease observed in the tested data that the D-psicose might also be effective when ingested for about 1 week, or when ingested in about a half amount. A significant decrease was observed in the uric acid level after two weeks from the ingestion. From the result of stratified analyses that the diabetes borderline patients showed a significant decrease after 2 weeks from the ingestion, it can be inferred that greater effects may be obtained particularly in pre-diabetic individuals.

The results for ALT are not presented because of a lack of significant decrease.

### EXAMPLE 2

### <Experiment Methods>

Four-week D-psicose ingestion tests were conducted with recruited healthy individuals (8 individuals with a fasting glucose level of 110 mg/dl) and diabetes borderline subjects (6 individuals with fasting glucose levels of 110 to 126 mg/dl, or HbA1c of 5.9 to 6.5%) (all subjects, males and females, were at least 20 years of age at the time of informed consent). Each subject ingested 15 g of D-psicose once per day.

The D-psicose was purchased from Izumoring Co., Ltd. The subjects were tested after 0, 2, and 4 weeks from the start of the ingestion, and after 4 weeks from the end of the ingestion period (week 8, post-ingestion observation). The subjects were fasted after the dinner served on the day before the testing date. The subjects were measured for γ-GTP, ALT, and ALP by using the service of BML Inc. A paired t-test was used for statistical comparison before and after the ingestion. In the figure, significant difference is given by *:0.05<P or **:0.01<P.

### <Results>

The results are shown in FIG. 2. γ-GTP, ALT, and ALP all significantly decreased after 2 weeks from the ingestion. These results demonstrated that the repeated ingestion of D-psicose (15 g/day) over the time period of about 2 weeks can clearly decrease γGTP, ALT, and ALP, and improve the biological function parameters in humans. It can be inferred from these results that ALT, which decreased after the 15 g/day ingestion in the test, requires about a 15 g of D-psicose. Stratified analyses of the healthy individuals and the diabetes borderline patients revealed that the former in particular showed a significant decrease (P<0.01) in γ-GTP and ALP after 2 weeks from the ingestion. It can be inferred from the extent of the decrease observed in the tested data that the D-psicose might also be effective when ingested for about 1 week, or when ingested in about a half amount.

### EXAMPLE 3

### <Experiment Methods>

Fourteen Wistar male rats (CLEA Japan), 3 weeks of age, were used for experiments in groups of seven. The animals were fed on solid feed CE-2 (CLEA Japan) for 1 week, and grouped according to body weight after this acclimation period. The basic feed compositions are presented in Table 1. The rare sugar-containing syrup was prepared by feeding a 10% (w/v) isomerized sugar solution (high fructose corn syrup) a strongly basic ion-exchange resin at a temperature of 60° C. (resin: Amberlite IRA900J [C1]). The sugar composition of the reaction solution was 40% D-glucose, 31% D-fructose, and 7% D-psicose. The remaining 22% were other carbohydrates, including D-mannose, and D-allose.

The rats were maintained for 8 weeks with free access to feed and drinking water. The animals were then anesthetized with ether, and the abdominal cavity was opened in turn. Blood was collected from the subrenal aorta in the abdomen with a heparin-treated syringe. For blood plasma measurement, AST (GOT), ALT (GPT), and the uric acid level were measured with commercially available kits. Experiment data are presented as mean value±standard error. The mean value significant difference between the groups was calculated by using unpaired t-test.

TABLE 1

| | | Rare sugar- |
|---|---|---|
| | Isomerized | containing |
| | sugar | syrup |
| Casein | 175.5 | 175.5 |
| Cornstarch | 285.2 | 285.2 |
| Isomerized sugar¹ | 407.5 | 135.9 |
| Rare sugar-containing syrup² | 0 | 271.7 |
| Cellulose | 43.9 | 43.9 |
| Mineral mixture (AIN-76) | 30.7 | 30.7 |
| Vitamin mixture (AIN-76) | 8.8 | 8.8 |
| DL-methionine | 2.6 | 2.6 |
| Choline chloride | 1.8 | 1.8 |
| Powdered oil | 43.9 | 43.9 |
| Butylhydroxytoluene | 0.1 | 0.1 |
| | 1000 | 1000 |

### <Results>

As shown in Table 2, a significant decrease was observed in AST, ALT, and the uric acid level after the ingestion of the rare sugar-containing syrup.

This result demonstrated that the D-psicose-containing composition prepared by reisomerization of isomerized sugar with alkali had the tendency to improve AST, ALT, and the uric acid level.

TABLE 2

| | | | Rare sugar- |
|---|---|---|---|
| | | Isomerized | containing |
| | | sugar | syrup |
| AST | (Karmen unit) | 78.6 ± 8.0 | 55.3 ± 4.3* |
| ALT | (Karmen unit) | 21.1 ± 1.9 | 13.8 ± 1.0** |
| Uric acid | (mg/dL) | 20.7 ± 0.4 | 18.5 ± 0.7* |
| level | | | |

### INDUSTRIAL APPLICABILITY

The present invention is applicable for improving biochemical biofunction parameters. With the use of the composition for improving biometabolism parameters, specifically the biometabolism parameter improving agent of the present invention, it is possible to improve biometabolism parameters (γ-GTP, AST, ALT, ALP, and uric acid level) of liver function indicator components in a medical examination, which is a case of trouble for middle aged people. The present invention can thus contribute to health.

1. An agent comprising D-psicose as an active ingredient and for lowering at least one biometabolism parameter selected from a uric acid level and a liver function indicator component level,
the agent being continuously ingested in a dose of 5 g or more per intake in terms of D-psicose.

2. An agent comprising D-psicose as an active ingredient and for lowering at least one biometabolism parameter selected from a uric acid level and a liver function indicator component level, the D-psicose being an alkali-isomerized product of at least one of glucose, fructose, and isomerized sugar,
the agent being continuously ingested in a dose of 5 g or more per intake in terms of D-psicose.

3. The biometabolism parameter lowering agent according to claim 1 or 2, wherein the liver function indicator component is at least one selected from γ-GTP, AST, ALT, and ALP.

4. The biometabolism parameter lowering agent according to claim 1 or 2, wherein the agent is ingested in a daily dose of 15 g/day for 2 weeks in terms of D-psicose.

5. The biometabolism parameter lowering agent according to claim 1 or 2, wherein the agent is a liver function protecting agent or a liver function improving agent.

6. The biometabolism parameter lowering agent according to claim 1 or 2, wherein the agent is a beverage, a food, a feed, a medicinal product, a quasi drug, an oral composition, or a cosmetic for protecting or improving liver function, or a beverage or food additive for protecting or improving liver function.
